SEQUENCE LISTING (3 sequences)

SEQ ID NO: 1 - DNA, 17 bp, Artificial Sequence
  Description of Artificial SequenceIgM secretoryregion PCR amplification primer
nacagggtgggtttacc

SEQ ID NO: 2 - DNA, 19 bp, Artificial Sequence
  Description of Artificial Sequencehuman VH3 V-region PCR amplification primer
gtttgcaagtgtccagtgt

SEQ ID NO: 3 - DNA, 21 bp, Artificial Sequence
  Description of Artificial Sequencehuman JH V-region PCR amplification primer
ngaggagacggtgaccagggt

(19) United States
(12) Patent Application Publication
(10) Pub. No.: US 2009/0298048 A1
(43) Pub. Date: Dec. 3, 2009

(54) NON-FLUORESCENT, NON-ENZYMATIC, CHEMILUMINESCENT AQUEOUS ASSAY

(75) Inventors: Vaughn V. Smider, Alameda, CA (US); William Heriot, Las Vegas, NV (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: INTEGRIGEN, INC., Notato, CA (US)

(21) Appl. No.: 10/578,054

(22) PCT Filed: Nov. 2, 2004
(86) PCT No.: PCT/US04/36577

§ 371 Date: Jun. 28, 2007

Related U.S. Application Data
(60) Provisional application No. 60/517,047, filed on Nov. 3, 2003.

(51) Int. Cl. C12Q 1/70; G01N 21/76; G01N 33/566; C12Q 1/68; C40B 40/08

(52) U.S. Cl. 435/5; 436/172; 436/501; 435/6; 506/17

(57) ABSTRACT

This invention provides for nonfluorescent, nonenzymatic, chemiluminescent aqueous assays in which the binding of two ligands is determined by a water soluble label system that emits light upon contact with a chemical energy transferring composition.

### CROSS-REFERENCE TO RELATED APPLICATIONS

[Not Applicable]

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention embodies a nonfluorescent, nonenzymatic, chemiluminescent aqueous assay in which the binding of two ligands is determined by a soluble label system that emits light upon contact with a chemical energy transferring composition.

### BACKGROUND OF THE INVENTION

DNA, protein, and other biomolecule detection and quantitation serves many different areas, ranging from basic research to clinical diagnostics, and drug discovery to forensics. The explosion of genomics and proteomics over the past decade has further highlighted the importance of simple, high-throughput, and sensitive detection technologies. Most biochemical detection mechanisms rely on detecting spectral characteristics of the biomolecules themselves, or to compounds to which they bind. Thus, calorimetric, fluorescent, chemiluminescent, and radiation properties are the principle mechanisms by which biomolecules are detected. Fluorescent dyes are used in several standard techniques, such as DNA sequencing, microarray analysis, fluorescence activated cell sorting (FACS), enzyme linked immunosorbant assays (ELISAs), and standard recombinant DNA techniques such as staining nucleic acids in agarose gel electrophoresis or dot blots.

### Fluorescence

Fluorescence mechanisms offer sensitive and often specific signals when applied to various biological and biochemical systems. Additionally, multiple fluorescent probes can be applied simultaneously in spacially addressable systems like microarrays (Fodor, et al. Science 251: 767-773 (1991)) or in single vessels (i.e. flow cytometry) to gather several bits of information in a single experiment. However, fluorescence systems have drawbacks that include the spectral limitations inherent in the dyes being used, background signal due to the excitation light, and expensive equipment such as laser light sources for highly sensitive applications.

Highly sensitive fluorescence techniques can detect as little as 10⁻¹² M of fluorophore. In microarray expression analysis, however, statistically relevant detection of a few copies of a gene may still be obscured by background signals. The sensitivity of fluorescence is generally limited by background and not by detection limitations. Sources of background include Raman emission, scattered light, impurities, and background luminescence (Slavik, CRC Press; Boca Raton (1993)). In complex biological systems, there is also the potential for decreased fluorescent signal due to shielding effects by molecules nearer to the light source.

### Chemiluminescence

Chemiluminescent and radiation detection mechanisms can be very sensitive and are often used in nucleic acid detection by hybridization as in southern and northern blots, or protein detection in western blots or ELISAs. However, radiation and current chemiluminescence mechanisms cannot easily be used to simultaneously measure multiple components in the way that fluorescence can (i.e. multiplexing), and can be expensive and/or only applicable in certain settings.

Two of the most widely used chemiluminescence systems in biological assays are the enhanced chemiluminescence assays for horseradish peroxidase, and the light emitting luciferase/luciferin reaction. Enhanced chemiluminescence relies on the reaction of horseradish peroxidase with H₂O₂ and luminol, a cyclic diacyl hydride, in the presence of an “enhancer” compound such as a phenol, naphthol, aromatic amine or benzothiazole. Oxidation and cleavage of the luminol ring structure results in formation of N₂ and emission of a photon. The spectral distribution of the emitted energy is broad (from 350 to 550 nm), and the emitting compound is free in solution (Kricka, et al. Luminescence Techniques in Chemical and Biochemical Analysis Marcel Dekker, Inc. 12: New York (1991)). Luciferase is an oxidase which oxidizes the luciferin molecule with concomitant production of a photon. In both systems, the reaction product is free in solution, which renders multiplex or spacially addressed applications (like microarrays) difficult. Another enzyme based system allows cleavage of an oxalate ester by alkaline phosphatase to produce a 1,2-dioxetane that intramolecularly transfers electrons to a substituent that produces a photon (Akhavan-Tafti, et al. Lumigen, Inc. U.S.A. 6,296,787 (2001)). Thus, all current chemiluminescent systems in biological detection utilize an enzyme with a chemiluminescent precursor.

Newer approaches for the detection of nucleic acid hybridizations and protein-nucleic acid interactions typically rely on energy transfer between a fluorophore and a quencher molecule or a second fluorophore (e.g., a fluorescence resonance energy transfer system). Thus, for example, a lumazine derivative has been used in conjunction with a bathophenanthroline-ruthenium complex as an energy transfer system in which the lumazine derivative acted as an energy donor and the ruthenium complex acted as an energy receptor. The lumazine derivative and ruthenium complex were attached to different nucleic acids. Energy transfer occurred when the two compounds were brought into proximity resulting in fluorescence. The system provided a mechanism for studying the interaction of molecules bearing the two groups (see, e.g., Bannwarth et al., Helvetica Chimica Acta. (1991) 74: 1991-1999, Bannwarth et al. (1991), Helvetica Chimica Acta. 74: 2000-2007, and Bannwarth et al., European Patent Application No. 0439036A2).

Another approach utilizes nucleic acid probes bearing a fluorophore and a quencher molecule. The probes were self-complementary and adopted a hairpin conformation in solution. The hairpin juxtaposed the fluorophore and the quencher thereby reducing or eliminating fluorescence of the fluorophore. When the probes hybridized to a target nucleic acid, they linearized, separating the fluorophore from the quencher molecule and thereby providing a fluorescent signal (see Tyagi and Kramer et al. (1996) Nature Biotechnology, 14: 303-308).

### SUMMARY OF THE INVENTION

This invention provides for a non-enzymatic, non-fluorescent, chemiluminescent method of detecting the interaction between two binding pair members by: (i) attaching a fluorophore to one of the two binding partner members; (ii) immobilizing the unlabeled binding pair member to a solid support; (iii) allowing the two binding pair members to bind to each other; and (iv) contacting the binding pair members with a solution comprising a chemical-energy transferring composition under conditions that stimulate the release of light energy from the fluorophore to allow detection of the interaction between the two binding pair members. The chemical-energy transferring composition may comprises an oxalic type compound such as those selected from the group consisting of: an oxalate ester, an oxalic thioester, an oxalate amide, a phosphate containing oxalic type compound. The oxalic type compound may contain electronegative substituents such as halogen atoms including chlorine.

Fluorophores of use in this invention are selected from one of the following groups: xanthenes, coumarins, benzimides, phenanthridines, acridines, cyanines, bodipy dyes, carbazole dyes, phenoxazine dyes, porphyrins, quinolines, and polycyclic aromatic hydrocarbons containing at least three fused rings, and quantum dots. The fluorophore may comprises a parent heteroaromatic ring system including but not limited to a parent xanthene ring. Other examples include: a rhodamine-type parent xanthene ring or a fluorescein-type xanthene ring.

Binding pair members are may be selected from the group consisting of: an antibody and antigen, two complementary nucleic acids, a protein and nucleic acid, a virus and host receptor, and a hormone and its cognate receptor.

The solid supports are selected from the group consisting of an addressed microarray, a bead, a gel and a transparent surface.

When used for sequencing a target nucleic acid the method comprises: (i) copying the target nucleic acid using a polymerase and nucleotide tripliosphates; (ii) randomly terminating polymerase activity using four polymerase blocking nucleotide inhibitors bearing a fluorophore specific for that inhibitor where the inhibitors are present in concentrations able to yield polymerase products terminated at different lengths; (iii) size fractionating the polymerase products in a gel; and, (iv) contacting the products with a solution comprising an oxalic type compound under conditions that stimulate the release of light energy from the fluorophore to allow detection of the product within the gel to sequence the nucleic acid.

The invention further provides for a non-enzymatic non-fluorescent chemiluminescent system for detecting the interaction between two binding pair members said system comprising an immobilized binding pair member and a non-immobilized binding pair member labeled with a fluorophore and a solution comprising a chemical energy transferring composition that is photo-reactive with the fluorophore. The system further encompasses the same embodiments set forth above for the methods.

The invention further provides for a system comprising a nucleic acid labeled with a fluorophore where the nucleic acid is in a gel and where the gel is infused with solution comprising an a chemical energy transferring composition that is photo-reactive with the fluorophore. This nucleic acid based system further encompasses the same embodiments set forth above for the methods.

The invention further provides for a system for detecting a biological composition comprising a biological composition labeled with a fluorophore where the composition is bound to a solid support and where the solid support is contacted with a solution comprising an oxalic type compound and a hydroperoxide. This system further encompasses the same embodiments set forth above for the methods.

Finally this invention provides for novel chemical energy transferring mixtures comprising: a) an oxalic type compound of the formula Z(CO)2Z; b) a peroxide component; and c) a biomolecule where the Z independently represents (can be the same or different) one of the following atoms: an oxygen, a sulfur, a nitrogen, a phosphorus. Preferred oxalate compounds are as described above. The biomolecule is selected from: a polynucleotide, an oligonucleotide, a peptide, a polypeptide, a polysaccharide. The peroxide component is hydrogen peroxide.

### BRIEF DESCRIPTION OF THE DRAWINGS

FIG. 1. Photon emitting systems. In fluorescence (top), incident light activates a fluorophore that emits a photon at a longer wavelength. Current chemiluminescence systems (middle) use enzymes to excite a precursor compound that then emits photons. Universal chemiluminescence uses a standard chemical activator to excite a fluorescent dye. In this system an oxalic type compound in the presence of hydroperoxide is believed to produce a dioxetane intermediate, which transfers energy to the fluorophore.

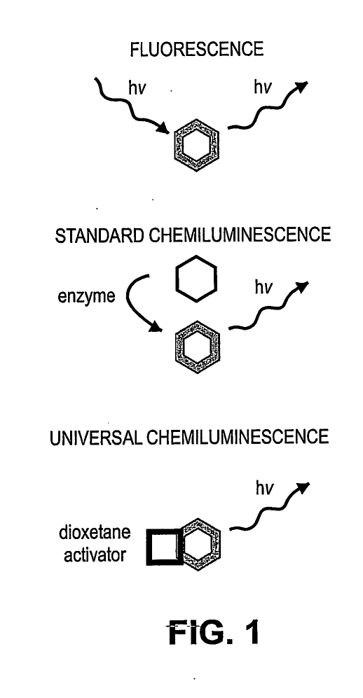

FIG. 2. Direct comparison of fluorescence to chemiluminescence. The same concentrations of rhodamine were either activated by light at 544 nm (squares) or chemical activator (triangles) and emission measured at 590 nm. The data from the experiment on the left was replotted using a log scale on the y-axis (middle). Data from the background wells containing no dye are expressed as RFU on the right with F indicating the fluorescence measurement and C indicating the chemiluminescence. Error bars are +2.5 SD of duplicate wells.

FIG. 3. Simultaneous activation of two dyes, with specific detection of one. Xanthoglow (diamonds and top row) and rhodamine were simultaneously activated by activated oxalate ester and measured at the emission wavelength for xanthoglow.

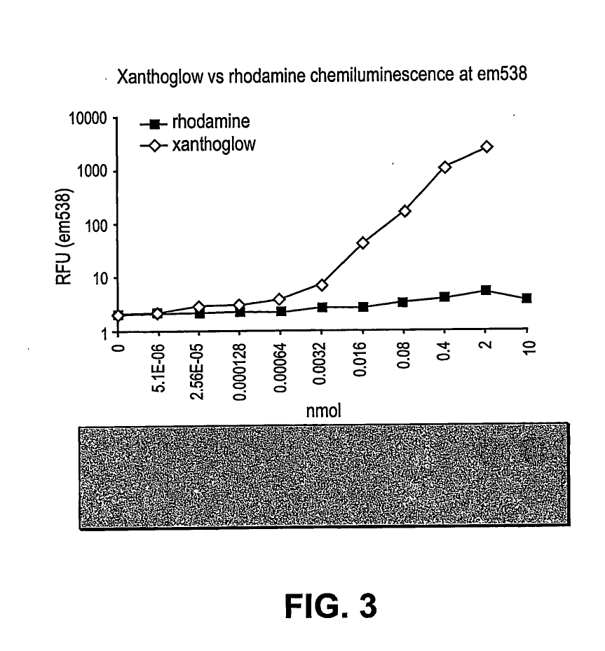

### DETAILED DESCRIPTION

### Introduction

The invention described herein embodies a chemiluminescent energy transferring (CET) mixture, and its uses to detect biomolecules. As described above, methods to detect biomolecules rely on inherent spectral characteristics of the biomolecule (e.g. the absorption maximum of double stranded DNA at 260 nm), or the labeling of the biomolecule with fluorescent, chemiluminescent, or radioactive molecules. There are several problems to these techniques with regards to background or when one wishes to simultaneously detect multiple molecules. Although fluorescence is highly sensitive, it relies on an incident light source of a particular wavelength. This property limits the use of certain fluorescent dyes due to excitation device constraints (e.g. the necessity for multiple lasers or filters). Additionally, fluorescence produces background signal due to light scatter and Raman effects. Standard chemiluminescent techniques, while sensitive, are limited in scope to detecting a single species of biomolecule. This is due to the property of these chemiluminescent molecules wherein the emitted photons comprise a broad wavelength. Thus separate emission wavelengths cannot be used for detection in standard chemiluminescence systems.

In many applications it is advantageous to employ multiple spectrally distinguishable fluorescent labels in order to achieve independent detection of a plurality of spatially overlapping analytes, i.e., multiplex fluorescent detection. Examples of methods utilizing multiplex fluorescent detection include single-tube multiplex DNA probe assays, PCR, single nucleotide polymorphisms and multi-color automated DNA sequencing. The number of reaction vessels may be reduced thereby simplifying experimental protocols and facilitating the production of application-specific reagent kits. In the case of multi-color automated DNA sequencing, multiplex fluorescent detection allows for the analysis of multiple nucleotide bases in a single electrophoresis lane thereby increasing throughput over single-color methods and reducing uncertainties associated with inter-lane electrophoretic mobility variations.

Assembling a set of multiple spectrally distinguishable fluorescent labels useful for multiplex fluorescent detection is problematic. Multiplex fluorescent detection imposes at least six severe constraints on the selection of component fluorescent labels, particularly for applications requiring a single excitation light source, an electrophoretic separation, and/or treatment with enzymes, e.g., automated DNA sequencing. First, it is difficult to find a set of structurally similar dyes whose emission spectra are spectrally resolved, since the typical emission band half-width for organic fluorescent dyes is about 40-80 nanometers (nm). Second, even if dyes with non-overlapping emission spectra are identified, the set may still not be suitable if the respective fluorescent quantum efficiencies are too low. Third, when several fluorescent dyes are used concurrently, simultaneous excitation becomes difficult because the absorption bands of the dyes are usually widely separated. Fourth, the charge, molecular size, and conformation of the dyes must not adversely affect the electrophoretic mobilities of the analyte. Fifth, the fluorescent dyes must be compatible with the chemistry used to create or manipulate the analyte, e.g., DNA synthesis solvents and reagents, buffers, polymerase enzymes, ligase enzymes, and the like. Sixth, the dye must have sufficient photostability to withstand laser excitation.

The present invention eliminates the excitation light source used by fluorescence, and replaces the excitation light energy with chemical energy. Thus, standard fluorescent dyes are applicable in the instant invention, however their mode of activation relies on the presence of a CET to directly transfer chemical energy to the fluorophore, thereby exciting it, and causing said fluorophore to emit photons at its characteristic wavelength (e.g. the same, or nearly the same, wavelength as they would emit if excited by light). Additionally, the instant invention differs from other chemiluminescent techniques in that multiple emission wavelengths are possible due to the use of a single CET with multiple fluorophores. Also, since the CET can be present in vast excess over the fluorophore, a single fluorophore molecule can be activated multiple times, producing a far greater signal per mole of label. In standard chemiluminescence, the chemiluminescent molecule is no longer active after it emits a photon. Thus the CET methods and compositions described herein combine the useful properties of fluorescence and chemiluminescence, and eliminate some of their drawbacks.

### DEFINITIONS

As used herein, the term “array” refers to an ordered spatial arrangement, particularly an arrangement of immobilized biomolecules or polymeric anchoring structures.

As used herein, the term “addressable array” refers to an array wherein the individual elements have precisely defined x and y coordinates, so that a given element at a particular position in the array can be identified.

As used herein, the term “binding pair members” refers to complementary biomolecules which can bind one another. Examples include antigens that bind antibodies, oligonucleotides that bind complimentary oligonucleotides, and ligands that bind receptors.

A “biomolecule” as used herein includes any member of a chemical class of compounds that can derive from a living organism. Examples of biomolecules include a protein, peptide, peptidomimetic, glycoprotein, proteoglycan, lipid, glycolipid, nucleic acid, carbohydrates, and the like as well as combinations of these molecules. As used herein, a biomolecule also includes chemical derivatives of molecules that can derive from a living organism.

As used herein, “Chemical energy transferring composition”, abbreviated “CET”, refers to a chemical mixture which can specifically transfer energy to a second molecule causing said second molecule to become electronically excited and subsequently to release a photon thereby emitting visible, infrared, or ultraviolet light. Thus, biological detection with a CET involves its direct conversion of chemical energy to light energy emitted from a second compound. As used herein, a CET refers to the mixture of an oxalic type compound and a peroxide component (as defined infra).

As used herein, the term “conjugated” refers to a stable attachment, which can be a covalent attachment or a noncovalent attachment, provided the noncovalent attachment is stable under the condition to which the bond is to be exposed. In particular, a polypeptide can be conjugated to a solid support through a linker, which can provide a non-cleavable, cleavable or reversible attachment.

The term “diluent” as used herein, is defined as solvent or vehicle, which does not cause insolubility of a CET, or any of the ingredients of the peroxide component, and in which the fluorophore is at least partially soluble.

The term “hydrogen peroxide compound” includes both hydrogen peroxide and hydrogen peroxide producing compounds. The term “peroxide component,” as used herein, means a solution of a hydrogen peroxide compound, a hydroperoxide compound, or a peroxide compound in a suitable diluent.

A “fluorophore” as defined herein is any dye that emits electromagnetic radiation of longer wavelength by a fluorescence mechanism upon irradiation by a source of electromagnetic radiation, including but not limited to a lamp, a photodiode or a laser.

As used herein, “quantum dot” refers to a fluorescent label comprising water-soluble semiconductor nanocrystal(s). One unique feature of a quantum dot is that its fluorescent spectrum is related to or determined by the diameter of its nanocrystals(s). Generally, quantum dots can be prepared which result in relative monodispersity; e.g., the diameter of the core varying approximately less than 10% between quantum dots in the preparation. Details of quantum dots and how they can be incorporated into microbeads may be found in the literatures, for example, in the articles by Chan and Nie, Science, 281:2016 (1998) and by Han et al., Nature Biotehnology, 19:631-635 (2001).

The term “solid-support” refers to a material in the solid-phase that interacts with reagents in the liquid phase by heterogeneous reactions. Solid-supports can be derivatized with proteins such as enzymes, peptides, oligonucleotides and polynucleotides by covalent or non-covalent bonding through one or more attachment sites, thereby “immobilizing” the protein or nucleic acid to the solid-support Examples of solid-support matrices include polystyrene, polyethylene, polyacrylamide, polypropylene, polyamide, Merrifield resin, sepharose, agarose, polydivinylbenzene, cellulose, alginic acid, chitosan, chitin, polystyrene-benzhydrylamine resin, an acrylic ester polymer, a lactic acid polymer, silica, silica gel, amino-functionalized silica gel, alumina, clay, zeolite, glass, controlled pore glass, or montmorillonite.

A “Non-enzymatic” mechanism refers to a chemical reaction which can occur in the absence of a protein or nucleic acid catalyst.

A “Non-Fluorescent” process is one wherein excitation photons are not required to produce emission photons. Thus, a fluorophore can participate in a non-fluorescent process by emitting photons in the absence of an excitation light source (e.g. by absorbing energy from a CET).

A “Nucleic-acid-modifying enzyme” refers to an enzyme that covalently alters a nucleic acid.

A “Polymerase” refers to an enzyme that performs template-directed synthesis of polynucleotides.

The term “nucleotide” refers to the phosphate ester of a nucleoside. The term “nucleoside” refers to a molecule comprising the covalent linkage of a pyrimidine or purine to a pentose ring (such as ribose or deoxyribose). The term “base” refers to a component of nucleic acid consisting of either adenine, guanine, thymine, cytosine, or uracil. Additionally, “purine” refers to either adenine or guanine, and “pyrimidine” refers to either thymine, cytosine, or uracil. The term “polynucleotide(s)” refers to a molecule containing at least one 5′ hydroxyl of one nucleotide covalently linked to one 3′ hydroxyl of at least one other nucleotide through a bond such as a phosphodiester bond.

In general, the terms used herein to describe the present invention rely on definitions as understood and used by those skilled in the art. In particular, chemical structures and substructures are described according to IUPAC recommendations (“Nomenclature of Organic Compounds: A Guide to IUPAC Recommendations 1993, R. Panico, W. H. Powell, and Jean-Claude Richer, Eds., Blackwell Science, Ltd., Oxford, U.K.).

Where substituent groups are specified by their conventional chemical formulae, written from left to right, they equally encompass the chemically identical substituents which would result from writing the structure from right to left, e.g., —CH₂ O— is intended to also recite —OCH₂—; —NHS(O)₂— is also intended to represent. —S(O)₂ HN—, etc.

Some of the compounds described herein contain one or more asymmetric centers and may thus give rise to enantiomers, diastereomers, and other stereoisometric forms that may be defined, in terms of absolute stereochemistry, as (R)- or (S)- or, as (D)- or (L)- for amino acids. The present invention is meant to include all such possible diastereomers, as well as, their racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. Likewise, all tautomeric forms are also intended to be included.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

Certain compounds of the present invention can exist in unsolvated forms as well as solvated forms, including hydrated forms. Except where noted, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

A “chemical group” is an atom or assemblage of atoms and organic chemical groups include but are not limited to alky, alkenyl, alkynyl, alkoxy, aryl, alkylaryl, heterocycle including heteroaryl, amide, thioamide, ester, amine, ether, thioether, halo, imine, cyano, nitro, carboxy, keto, aldehydro, and combinations thereof.

An “oxalic” type compound refers to a compound with two immediately adjacent carbonyl groups connected by a single carbon-carbon bond.

“Parent Heteroaromatic Ring System” refers to a parent aromatic ring system in which one or more carbon atoms (and any necessary associated hydrogen atoms) are each independently replaced with the same or different heteroatom. Typical heteratoms to replace the carbon atoms include, but are not limited to, N, P, O, S, Si, etc. Specifically included within the definition of “parent heteroaromatic ring systems” are fused ring systems in which one or more rings are aromatic and one or more of the rings are saturated or unsaturated, such as, for example, arsindole, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Parent Xanthene Ring” refers to a heteroaromatic ring system of a type typically found in the xanthene class of fluorescent dyes (which includes rhodamine and fluorescein dyes, defined infra), i.e., a heteroaromatic ring system having the general structure:

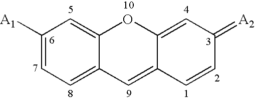

In the parent xanthene ring depicted above, A¹ is —OH or —NH₂ and A₂ is ═O or ═NH₂⁺. Wien A¹ is —OH and A² is ═O, the parent xanthene ring is a fluorescein-type parent xanthene ring, which is defined in more detail, infra. When A¹ is —NH₂ and A² is ═NH₂⁺, the parent xanthene ring is a rhodamine-type parent xanthene ring, which is defined in more detail, infra. When A¹ is —NH₂ and A² is ═O, the parent xanthene ring is a rhodol-type parent xanthene ring. In the parent xanthene ring depicted above, one or both nitrogens of A¹ and A² (when present) and/or one or more of the carbon atoms at positions C1, C2, C4, C5, C7 and C8, can be independently substituted with a wide variety of the same or different substituents, as is well known in the art. Typical substituents include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O—, —S(O)₂OH, —S(O)₂R, —C(O)R, —C(O)X, —C(S)R, —C(S)X, —C(O)OR, —C(O)O—, —C(S)OR, —C(O)SR, —C(S)SR, —C(O)NRR, —C(S)NRR and —C(NR)NRR, where each X is independently a halogen (preferably —F or —Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, heteroaryl, 6-26 membered heteroarylalkyl or 5-20 membered heteroaryl-heteroaryl. Moreover, the C1 and C2 substituents and/or the C7 and C8 substituents can be taken together to form substituted or unsubstituted (C₅-C₂₀) aryleno bridges. Generally, substituents groups which do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —F, —Br, —CN and —CF₃.

When A¹ is —NH₂ and/or A² is ═NH₂, the xanthene nitrogens can be included in bridges involving the same nitrogen atom or adjacent carbon atoms, e.g., (C₁-C₁₂) alkyldiyl, (C₁-C₁₂) alkyleno, 2-12 membered heteroalkyldiyl and/or 2-12 membered heteroalkyleno bridges.

Any of the substituents substituting carbons C1, C2, C4, C5, C7 or C8 and/or the xanthene nitrogen atoms (when present) can be further substituted with one or more of the same or different substituents, which are typically selected from the group consisting of —X, —R′, ═O, —OR′, —SR, ═S, —NR′R′, ═NR′, —CX₃, —CN, —OCN, —SCN, —NCO, —NCS, —NO, —NO₂, ═N₂, —N₃, —NHOH, —S(O)₂O⁻, —S(O)₂OH, —S(O)₂R′, —P(O)(O—)₂, —P(O)(OH)₂, —C(O)R′, —C(O)X, —C(S)R′, —C(S)X, —C(O)OR′, —C(O)O⁻, —C(S)OR′, —C(O)SR, —C(S)SR, —C(O)NR′R′, —C(S)NR′R′ and —C(NR)NR′R′, where each X is independently a halogen (preferably —F or —Cl) and each R′ is independently hydrogen, (C₁-C₆) alkyl, 2-6 membered heteroalkyl, (C₅-C₁₄) aryl or heteroaryl. Exemplary parent xanthene rings include, but are not limited to, rhodamine-type parent xanthene rings and fluorescein-type parent xanthene rings, each of which is defined in more detail, infra.

“Rhodamine-Type Parent Xanthene Ring” refers to a parent xanthene ring in which A¹ is —NH₂ and A² is ═NH.sub.₂.sup.+, i.e., a parent xanthene ring having the general structure:

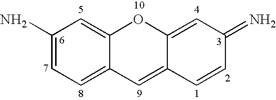

In the rhodamine-type parent xanthene ring depicted above, one or both nitrogens and/or one or more of the carbons at positions C1, C2, C4, C5, C7 or C8 can be independently substituted with a wide variety of the same or different substituents, as previously described for the parent xanthene rings. Exemplary rhodamine-type parent xanthene rings include, but are not limited to, the xanthene rings of the rhodamine dyes described in U.S. Pat. No. 5,936,087; U.S. Pat. No. 5,750,409; U.S. Pat. No. 5,366,860; U.S. Pat. No. 5,231,191; U.S. Pat. No. 5,840,999; U.S. Pat. No. 5,847,162; U.S. application Ser. No. 09/277,793, filed Mar. 27, 1999; PCT Publication WO 97/36960; PCT Publication WO 99/27020; Sauer et al., 1995, J. Fluorescence 5(3):247-261; Arden-Jacob, 1993, Neue Lanwellige Xanthen-Farbstoffe fur Fluoreszenzsonden und Farbstoff Laser, Verlag Shaker, Germany; and Lee et al., 1992, Nucl. Acids Res. 20(10):2471-2483. Also included within the definition of “rhodamine-type parent xanthene ring” are the extended-conjugation xanthene rings of the extended rhodamine dyes described in U.S. application Ser. No. 09/325,243, filed Jun. 3, 1999.

“Fluorescein-Type Parent Xanthene Ring” refers to a parent xanthene ring in which A¹ is —OH and A² is ═O, i.e., a parent xanthene ring having the structure:

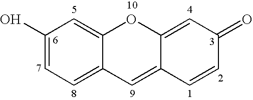

In the fluorescein-type parent xanthene ring depicted above, one or more of the carbons at positions C1, C2, C4, C5, C7 or C8 can be independently substituted with a wide variety of the same or different substituents, as previously described for the parent xanthene rings. Exemplary fluorescein-type parent xanthene rings include, but are not limited to, the xanthene rings of the fluorescein dyes described in U.S. Pat. No. 4,439,356; U.S. Pat. No. 4,481,136; U.S. Pat. No. 5,188,934; U.S. Pat. No. 5,654,442; U.S. Pat. No. 5,840,999; WO 99/16832; and EP 0 050 684. Also included within the definition of “fluorescein-type parent xanthene ring” are the extended xanthene rings of the fluorescein dyes described in U.S. Pat. No. 5,750,409 and U.S. Pat. No. 5,066,580.

“Xanthene Dye” or “xanthene” refers to a class of fluorescent dyes which Consist of a parent xanthene ring substituted at the xanthene C-9 carbon with a substituted phenyl ring or other, typically acyclic, substituent. Common substituted phenyl rings found in xanthene dyes include, e.g., 2-carboxyphenyl, dihalo-2-carboxyphenyl, tetrahalo-2-carboxyphenyl, 2-ethoxycarbonylphenyl, dihalo-2-ethoxycarbonylphenyl and tetrahalo-2-thoxycarbonylphenyl. Common acyclic substituents found in xanthene dyes include, e.g., carboxyethyl and perfluoroalkyl (eg., trifluoromethyl, pentafluoroethyl and heptafluoropropyl). Typical xanthene dyes include the fluorescein dyes and the rhodamine dyes, which are described in more detail, infra

“Rhodamine Dye” refers to the subclass of xanthene dyes in which the xanthene ring is a rhodamine-type parent xanthene ring. Typical rhodamine dyes include, but are not limited to, rhodamine B, 5-carboxyrhodamine, rhodamine X (ROX), 4,7-dichlororhodamine X (dROX), rhodamine 6G (R6G), rhodamine 110 (R110), 4,7-dichlororhodamine 110 (dR110), tetramethyl rhodamine (TAMRA) and 4,7-dichloro-tetramethylrhodamine (dTAMRA). Additional typical rhodamine dyes can be found, for example, in U.S. Pat. No. 5,936,087; U.S. Pat. No. 5,750,409; U.S. Pat. No. 5,366,860; U.S. Pat. No. 5,231,191; U.S. Pat. No. 5,840,999; U.S. Pat. No. 5,847,162; U.S. application Ser. No. 09/038,191, filed Mar. 10, 1998; U.S. application Ser. No. 09/277,793, filed Mar. 27, 1999; U.S. application Ser. No. 09/325,243, filed Jun. 3, 1999; PCT Publication WO 97/36960; PCT Publication WO 99/27020; Sauer et al., 1995, J. Fluorescence 5(3):247-261; Arden-Jacob, 1993, Neue Lanwellige Xanthen. Farbstoffe fur Fluoreszenzsonden und Farbstoff Laser, Verlag Shaker, Germany; and Lee et al., 1992, Nucl. Acids Res. 20(10):2471-2483.

“Fluorescein Dye” refers to the subclass of xanthene dyes in which the parent xanthene ring is a fluorescein-type parent xanthene ring. Typical fluorescein dyes include, but are not limited to, 5-carboxyfluorescein (5-FAM), 6-carboxyfluorescein (6-FAM). Additional typical fluorescein dyes can be found, for example, in U.S. Pat. No. 5,750,409; U.S. Pat. No. 5,066,580; U.S. Pat. No. 4,439,356; U.S. Pat. No. 4,481,136; U.S. Pat. No. 5,188,934; U.S. Pat. No. 5,654,442; U.S. Pat. No. 5,840,999; PCT publication WO 99/16832; EP 0 050 684; and U.S. application Ser. No. 08/942,067, filed Oct. 1, 1997.

The term “electron withdrawing” or “electronegative” denotes the tendency of a substituent to attract valence electrons of the molecule of which it is a part, i.e., an electron-withdrawing substituent is electronegative (e.g. a halogen or a nitro group).

“Parent Reteroaromatic Ring System:” refers to a parent aromatic ring system in which one or more carbon atoms (and any necessary associated hydrogen atoms) are each independently replaced with the same or different heteroatom. Typical heteratoms to replace the carbon atoms include, but are not limited to, N, P, O, S, Si, etc. Specifically included within the definition of “parent heteroaromatic ring systems” are fused ring systems in which one or more rings are aromatic and one or more of the rings are saturated or unsaturated, such as, for example, arsindole, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Alkyl” is intended to include aliphatically saturated linear or branched, hydrocarbon structures and combinations thereof “Lower alkyl” means alkyl groups of from 1 to 8 carbon atoms. Examples of lower alkyl groups include methyl, ethyl, propyl, isopropyl, butyl, s- and t-butyl, pentyl, hexyl, octyl, and the like. Preferred alkyl groups are those of C20 or below, particularly C10 or below.

“Cycloalkyl” includes cyclic hydrocarbon groups of from 3 to 8 carbon atoms. Examples of lower cycloalkyl groups include c-propyl, c-butyl, c-pentyl, norbornyl, decalin, and the like, and may be aliphatically saturated or unsaturated.

“Alkenyl” includes C₂-C₈ unsaturated hydrocarbons of a linear or branched configuration and combinations thereof. Examples of alkenyl groups include vinyl, allyl, isopropenyl, pentenyl, hexenyl, 1-propenyl, 2-butenyl, 2-methyl-2-butenyl, 2,4-hexadienyl and the like.

“Alkynyl” includes C₂-C₈ hydrocarbons of a linear or branched configuration and combinations thereof containing at least one carbon-carbon triple bond. Examples of alkynyl groups include ethyne, propyne, butyne, pentyne, 3-methyl-1-butyne, 3,3-dimethyl-1-butyne and the like.

“Alkoxy” refers to groups of from 1 to 8 carbon atoms of a straight, branched, cyclic configuration and combinations thereof. Examples include methoxy, ethoxy, propoxy, isopropoxy, cyclopropyloxy, cyclohexyloxy and the like.

“Acylamino” refers to acylamino groups of from 1 to 8 carbon atoms of a straight, branched or cyclic configuration and combinations thereof. Examples include acetylamino, butyrylamino, cyclohexylanoylamino, and the like.

“Hydrocarbylamino” refers to a moiety consisting of hydrogen and carbon bonded to nitrogen and of from about 1 to 8 carbon atoms for each hydrocarbyl group, there being up to 4, usually 3, hydrocarbyl groups. By “hydrocarbyl is intended any molecule or core of a molecule composed solely of hydrogen and carbon.

“Halogen” includes F, Cl, Br, and I.

“Halophenyl” means phenyl substituted with 1-5 halogen atoms. Examples include pentachlorophenyl, pentafluorophenyl and 2,4,6-trichlorophenyl.

“Aryl” and “heteroaryl” mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S; each of which rings is optionally substituted with 1-3 lower alkyl, substituted alkyl, substituted alkynyl, .dbd.O, —NO.sub.2, halogen, hydroxy, alkoxy, OCH(COOH)₂, cyano, —NZZ, acylamino, phenyl, benzyl, phenoxy, benzyloxy, heteroaryl, or heteroaryloxy; each of said phenyl, benzyl, phenoxy, benzyloxy, heteroaryl, and heteroaryloxy is optionally substituted with 1-3 substituents selected from lower alkyl, alkenyl, alkynyl, halogen, hydroxy, alkoxy, cyano, phenyl, benzyl, benzyloxy, carboxamido, heteroaryl, heteroaryloxy, —NO₂ or —NZZ (wherein Z is independently H, lower alkyl or cycloalkyl, and -ZZ may be fused to form a cyclic ring with nitrogen).

The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

“Arylalkyl” means an alkyl residue attached to an aryl ring. Examples are benzyl, phenethyl and the like.

“Heteroarylalkyl” means an alkyl residue attached to a heteroaryl ring. Examples include, e.g., pyridinylmethyl, pyrimidinylethyl and the like.

“Heterocycloalkyl” means a cycloalkyl where one to two of the methylene (CH₂) groups is replaced by a heteroatom such as O, NZ′ (wherein Z is H or alkyl), S or the like; with the proviso that except for nitrogen when two heteroatoms are present, they must be separated by at least one carbon atom. Examples of heterocycloalkyl include tetrahydrofuranyl, piperidine, dioxanyl and the like.

“Alkylcarbonyl” means —C(O)R″, wherein R″ is alkyl.

“Substituted” alkyl, alkenyl, alkynyl, cycloalkyl, or heterocycloalkyl means alkyl, alkenyl, alkynyl, cycloalkyl, or heterocycloalkyl wherein up to three H atoms on each C atom therein are replaced with halogen, hydroxy, loweralkoxy, carboxy, carboalkoxy, carboxamido, cyano, carbonyl, —NO₂, —NZZ; alkylthio, sulfoxide, sulfone, acylamino, amidino, phenyl, benzyl, heteroaryl, phenoxy, benzyloxy, heteroaryloxy, or substituted phenyl, benzyl, heteroaryl, phenoxy, benzyloxy, or heteroaryloxy.

An “alkylaryl group” refers to an alkyl (as described above), covalently joined to an aryl group (as described above).

“Carbocyclic aryl groups” are groups wherein the ring atoms on the aromatic ring are all carbon atoms. The carbon atoms are optionally substituted.

“Heterocyclic aryl groups” are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted.

An “amide” refers to an —C(O)—NH—, where Z is either alkyl, aryl, alklyaryl or hydrogen.

A “thioamide” refers to —C(S)—NH-Z, where Z is either alkyl, aryl, alklyaryl or hydrogen.

An “ester” refers to an —C(O)—OZ′, where Z′ is either alkyl, aryl, or alklyaryl.

An “amine” refers to a—N(Z″)Z′″, where Z″ and Z′″, is independently either hydrogen, alkyl, aryl, or alklyaryl, provided that Z″ and Z′″ are not both hydrogen.

An “ether” refers to Z-O-Z, where Z is either alkyl, aryl, or alkylaryl.

A “thioether” refers to Z-S-Z, where Z is either alkyl, aryl, or alkylaryl.

A “cyclic molecule” is a molecule which has at least one chemical moiety which forms a ring. The ring may contain three atoms or more. The molecule may contain more than one cyclic moiety, the cyclic moieties may be the same or different.

A “linear molecule” does not contain a ring structure. However, the molecule may be straight or branched.

As used herein, the term “heteroatom” is meant to include oxygen (O), nitrogen (N), sulfur (S) and silicon (Si).

### Chemical Energy Transferring Mixtures

The object of this invention are chemical energy transferring mixtures obtained by admixing reactants including (1) an oxalic type compound, (2) a hydroperoxide, and (3) a diluent. In a preferred embodiment, an alkaline material is included where necessary, in an amount at least sufficient to obtain a pH of at least above pH 5 and below about pH 12. The oxalic type compound referred to above has a general structure of the formula:

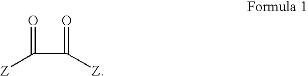

wherein Z contains one of the following atoms: oxygen, nitrogen, sulfur and phosphorus; and at least one electronegative group.

For example these may include the class of oxalic esters wherein Z contains an oxygen atom, and has the general formula:

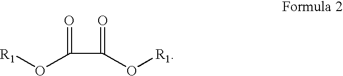

wherein R₁ preferably contains an electronegative group. Additionally, the oxalic type compound may be an oxalic thioester, wherein Z is a sulfur atom, and has the general formula:

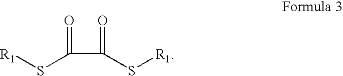

or may be an oxalic amide, wherein Z is a nitrogen atom, and has the general formula:

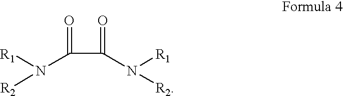

or may be oxalic phosphorus compounds, wherein Z is a phosphorus atom, and has the general formula:

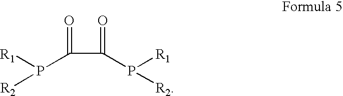

In the above example the phosphorus atom is in the trivalent state, The phosphorus atom may also be in the pentavalent state wherein it is additionally bound to a oxygen or sulfur atom.

In a preferred embodiment, one of the R groups in the above formulae should bear an electronegative substitutent. The electronegative substitutent being herein defined as a compound which includes atoms sufficiently electron attracting to make the parent (hydrogen substituted) compound at least as acidic as a pure hydrocarbon when compared under substantially similar conditions, such as in a common solvent of this invention.

The compositions for reaction with a peroxide component to generate chemiluminescence can contain any fluid diluent which solubilizes the compound of formula (1) to provide initial concentrations in the reacting system of about 10⁻⁴ M to 10 M, preferably about 10⁻² M to 1 M., of the compound of formula (I). The diluent must be relatively unreactive toward the compound of formula (I), the fluorphore, and the ingredients of the peroxide component.

Hydrogen peroxide is the preferred hydroperoxide and may be employed as a solution of hydrogen peroxide in a solvent or as an anhydrous hydrogen peroxide compound such as sodium perborate, sodium peroxide and the like. Whenever hydrogen peroxide is contemplated to be employed, any suitable compound may be substituted which will produce hydrogen peroxide.

The synthesis of the oxalic type compounds described above will depend on the type of compound desired, and the identity of the Z group in formula 1. The chemical transformations utilized and reaction methodology is well known and discussed in standard textbooks (Schmid Mosby St. Louis (1996)). Synthetic details for some of the oxalic type compounds are described in detail below in the “Examples” section.

### Fluorophores

As discussed above, a wide variety of fluorescent dyes may find application as the fluorophores in the subject invention. These dyes will fall into various classes, where combinations of dyes may be used within the same class or between different classes. Included among the classes are dyes such as the xanthene dyes, e.g. fluoresceins and rhodamines; coumarins, e.g. umbelliferone; benzimide dyes, e.g. Hoechst 33258, phenanthridine dyes; e.g. Texas Red and ethidium dyes; acridine dyes; Bodipy; cyanine dyes, such as thiazole orange, thiazole blue, Cy3, Cy 5, and Cyfr; carbazole dyes; phenoxazine dyes; porphyrin dyes; quinoline dyes; or the like. Thus, the dyes may absorb in the ultraviolet, visible or infra-red ranges. For the most part, the fluorescent molecules will have a molecular weight of less than about 2 kDal, generally less than about 1.5 kdal.

A fluorophore can also be comprised of a protein, such as green fluorescent protein (GFP). Tsien, et. al (U.S. Pat. No. 6,627,449) describes GFP and variants thereof.

Typical fluorophores for use in the present invention are those which have a spectral emission falling between 300 and 1200 nanometers and which are at least partially soluable in the diluent employed. Among these are the conjugated polycyclic aromatic compounds having at least three fused rings, such as anthracene, substituted anthracene, benzanthracene, phenanthracene, substituted phenanthracene, naphthacene, substituted naphthacene, pentacene, substituted pentacene, perylene, substituted perylene, and the like. Typical substitutents for all of these are phenyl, lower alkyl, halogen, cyano, alkoxy and other like substituents which do not interfere with the light generating reaction contemplated herein.

Numerous other fluorescent compounds having the properties given herein are well known in the art. Many of these are fully described in “Fluorescence and Phosphorescence” by Peter Pringsheim, Interscience Publishers, New York, N.Y. 1969. Other fluorescers are described in “The Colour Index,” Second Edition, Volume 2, The American Association of Textile Chemists and Colorists, 1956, pp 2907-2923. While only typical fluorescent compounds are listed herein, the person skilled in the art is fully aware of the fact that this invention is not so restricted, and that numerous other fluorescent compounds having similar properties are contemplated for use herein.

Several fluorophores are commonly used in the biological sciences and include derivatives of fluorescein, rhodamine, cyanine, and phycoerythrin.

Quantum dots have found their applications in bioanalysis just recently. Quantum dots have unique fluorescence properties based on their size (See e.g., Chan and Nie, Science, 281:2016 (1998); Han et al., Nature Biotehnology, 19:631-635 (2001); and U.S. Pat. No. 6,252,664). Quantum dot nanocrystals are nanometer scale particles that are neither small molecules nor bulk solids. Their composition and small size (a few hundred to a few thousand atoms) give these dots extraordinary optical properties, which can be readily customized by changing the size or composition of the dots. This property is the basis for encoding using quantum dots. Any suitable quantum dot can be used in the present beads. In a specific embodiment, the quantum dot used in the present beads comprises a Cd—X core, X being Se, S or Te. Preferably, the quantum dot can be passivated with an inorganic coating shell, e.g., a coating shell comprising Y-Z, Y being Cd or Zn, and Z being S or Se. Also preferably, the quantum dot can comprise a Cd—X core, X being Se, S or Te, a Y-Z shell, Y being Cd or Zn, and Z being S or Se, and the bead can further be overcoated with a trialkylphosphine oxide.

The subject fluorophores may be conjugated to a biomolecule using any convenient means. One conventional means for conjugation employ homobifunctional and heterobifunctional crosslinking reagents. Homobifunctional reagents carry two identical functional groups, whereas heterobifunctional reagents contain two dissimilar functional groups to link the biologics to the bioadhesive. A vast majority of the heterobifunctional cross-linking agents contain a primary amine-reactive group and a thiol-reactive group. Covalent crosslinking agents are selected from reagents capable of forming disulfide (S—S), glycol (—CH(OH)—CH(OH)—), azo (—N═N—), sulfone (—S(═O₂—), ester (—C(═O)—O—), or amide (—C(═O)—N—) bridges.

Additionally, a large number of nucleosides or amino acids are available, which are functionalized, and may be used in the synthesis of a polynucleotide or polypeptide, respectively. By synthesizing the subject nucleic acid or amino acid labels, one can define the specific sites at which the fluorophores are present. Commercially available synthesizers may be employed in accordance with conventional ways. Standard fluorescent labeling protocols for nucleic acids are described, e.g., in Sambrook et al.; Kambara, H. et al. (1988) BioTechnology 6:816-821; Smith, L. et al. (1985) Nuc. Acids Res. 13:2399-2412; for polypeptides, see, e.g., Allen G. (1989) Sequencing of Proteins and Peptides, Elsevier, N.Y., especially chapter 5, and Greenstein and Winitz (1961) Chemistry of the Amino Acids, Wiley and Sons, New York. Carbohydrate labeling is described, e.g., in Chaplin and Kennedy (1986) Carbohydrate Analysis: A Practical Approach, IRL Press, Oxford. Labeling of other polymers will be performed by methods applicable to them as recognized by a person having ordinary skill in manipulating the corresponding polymer.

### Binding Pair Assays

Detection of binding member pair interactions find use in a variety of applications, including various separation techniques, such as electrophoresis, chromatography, or the like, where one wishes to have optimized spectroscopic properties, high sensitivity and comparable influence of the labels on the migratory aptitude of the components being analyzed. Of particular interest is electrophoresis, such as gel, capillary, etc. Among chromatographic techniques are HPLC, affinity chromatography, thin layer chromatography, paper chromatography, and the like. Additionally, the fluorescent labels are particularly useful in the detection of binding pair interactions on arrays, such as oligonucleotide, polynucleotide, peptide, or polypeptide microarrays.

A variety of molecular structures can be used as the binding pair members. Examples of various classes of binding pair members are
(a) nucleic acids, including both RNA and DNA,
(b) modified nucleic acids, for example those where oxygen atoms are replaced by sulfur, carbon, or nitrogen atoms, and those where phosphate groups are replaced by sulfate groups, carboxylate group, or N-(2-aminoethyl)glycine,
(c) polypeptides,
(d) polysaccharides, and
(e) lipids
(f) groups that can be joined in stepwise manner, examples of which are di-functional groups such as haloamines.

Additionally binding pair members can be comprised of different classes. For example a binding pair member could comprise a nucleic acid and a polypeptide which can form a complex with one another. Alternatively, they can be of a single class; for example complementary nucleic acids capable of hybridizing to one another are exemplary binding pair members. Binding pair members can bind with any stoichiometry, i.e. a homodimer, heterodimer, heterotrimer, heterotetramer, etc. In this regard, two binding pair members could even be chemically identical to one another. For example the oligonucleotide ATGCAT is a palindrome capable of self binding, and thus fits the definition of a binding pair member. Additionally, binding pair members can optionally include additional components, such that a complex is formed between the two binding pair members and one or more additional components. In this regard, the binding pair members might not interact directly, but may be “bridged” by a third component which brings the binding pair members in proximity to one another.

Binding pair members can interact non-covalently or covalently. A catalyst may be additionally incorporated to facilitate the binding of binding pair members. For example, a DNA ligase can covalently join two nucleic acids, which are considered binding pair members.

Polymeric binding pair members are preferred. For many applications, classes of particular interest as binding pair members are that of nucleic acids, polypeptides, or polysaccharides. The bases within the oligonucleotides include those that are naturally occurring as well synthetic bases, plus bases that have been modified to facilitate the attachment of the fluorophores; naturally occurring bases (including modified analogues) are currently of greatest interest Oligonucleotide backbones contemplated in this invention are polymerized chains of monomeric units selected form purine and pyrimidine mononucleotides, hybridizing analogues of these mononucleotides, and all other structures listed above. Within this class, DNA and RNA are particularly preferred. The length of the oligonucleotide can vary considerably and is not critical to this invention.

Preferably one of the binding pair members should be conjugated to a fluorophore. Once a particular fluorophore has been selected, appropriate labeling protocols will be applied, as described above for specific embodiments.

In some embodiments, the target need not actually be labeled if a means for detecting where interaction takes place is available. As described below, for a nucleic acid embodiment, such may be provided by an intercalating dye which intercalates only into double stranded segments, e.g., where interaction occurs. See, e.g., Sheldon et al. U.S. Pat. No. 4,582,789.

### Types of Solid Supports

The support matrix is comprised of insoluble materials, preferably having a rigid or semi-rigid character, and may be any shape, e.g. spherical, as in beads, rectangular, irregular particles, resins, gels, microspheres, or substantially flat as in a microchip. In some embodiments, it may be desirable to create an array of physically separate regions on the support with, for example, wells, raised regions, dimples, pins, trenches, rods, pins, inner or outer walls of cylinders, and the like.

Preferred support materials include agarose, polyacrylamide, magnetic beads (Stamm, S. and Brosius, J. (1995) “Solid phase PCR” in PCR 2, A Practical Approach, IRL Press at Oxford University Press, Oxford, U.K., p. 55-70.), polystyrene (Andrus, et. al. Nucleic Acids Symp Ser. 1993; 29:5-6.), controlled-pore— glass (Caruthers, Science (1985) 230: 281-5.), polyacrylate hydroxethylmethacrylate, polyamide, polyethylene, polyethyleneoxy, or copolymers and grafts of such. Other solid-supports include small particles, membranes, frits, non-porous surfaces, addressable arrays, vectors, plasmids, or polynucleotide-immobilizing media. Additionally, fullerenes can conceivably be used as a solid support, as well as derivatized fullerenes such as gadolinium fullerenes which contain paramagnetic properties. In preferred embodiments addressable arrays, gels, and beads are used as described below.

Functional groups suitable for facilitating the attachment of a binding pair member can be incorporated into the polymer structure by conventional means, including the use of monomers that contain the desired functional group(s), either as the sole monomer or as a co-monomer. Examples of suitable functional groups are amine groups (—NH₂), ammonium groups (—NH₃⁺ or —NR₃⁺), hydroxyl groups (—OH), carboxylic acid groups (—COOH), isocyanate groups (—NCO), etc A useful monomer for introducing carboxylic acid groups into polyolefins, for example, is acrylic acid or methacrylic acid.

Attachment of the ligand to the microparticle can be achieved by electrostatic attraction, specific affinity interaction, hydrophobic interaction, or covalent bonding. Covalent bonding is preferred. Linking groups can be used as a means of increasing the density of reactive groups on the microparticle and of modulating steric hindrance to increase the range and sensitivity of the assay, or as a means of adding specific types of reactive groups to the microparticle to broaden the number of types of ligands that can be affixed to the microparticle. Examples of suitable useful linking groups are polylysine, polyaspartic acid, polyglutamic acid, polyarginine, etc.

### Assay Formats

Several assay formats are contemplated for the instant invention. Virtually any assay which utilizes a fluorophore that is detected through excitation by an exogenous light can also be utilized to allow excitation through a CET. Examples of biological assays which use fluorescence as a detection mechanism are: microarray analysis, gel electrophoresis, capillary electrophoresis, HPLC analysis, enzyme linked immunosorbant assays (ELISAS), flow cytometry (e.g. fluorescence activated cell sorting), fluorescence spectroscopy of analytes, and fluorescence microscopy.

Such assays may be heterogeneous or homogeneous, and they may be sequential or simultaneous. Heterogeneous assays, which rely in part on the transfer of analyte from a liquid sample to a solid phase by the binding of the analyte during the assay to the surface of the solid phase are particularly employed. In heterogeneous assay techniques, the reaction product is separated from excess sample, assay reagents and other substances by removing the solid phase from the reaction mixture. At some stage of the assay, whose sequence varies depending on the assay protocol, the solid phase and the liquid phase are separated and the determination leading to detection and/or quantitation of the analyte is performed on one of the two separated phases. One type of solid phase that has been used are magnetic particles, which offer the combined advantages of a high surface area and the ability to be temporarily immobilized at the wall of the assay receptacle by imposition of a magnetic field while the liquid phase is aspirated, the solid phase is washed, or both. Descriptions of such particles and their use are found in Forrest et al., U.S. Pat. No. 4,141,687 (Technicon Instruments Corporation, Feb. 27, 1979); Ithakissios, U.S. Pat. No. 4,115,534 (Minnesota Mining and Manufacturing Company, Sep. 19, 1978); Vlieger, A. M., et al., Analytical Biochemistry 205:1-7 (1992).

Of particular utility in the present invention is the ability to activate several fluorophores simultaneously, i.e. to “multiplex”. It is increasingly desirable to assay multiple different analytes simultaneously in the same sampling. Such “multiplexing” permits greater throughput, minimizes sample volume and handling, provides internal standardization control, decreases assay cost and increases the amount of information that is obtainable from each sample.

Various approaches for conducting multiplexed assays have been proposed. U.S. Pat. No. 6,319,668 (Nova, et al.), for example, employs computer-facilitated microarrays of reagents to conduct multiplexed analysis of multiple analytes. International Patent publication WO9926067A1 (Watkins et al.) describes the use of magnetic particles that vary in size to assay multiple analytes; particles belonging to different distinct size ranges are used to assay for different analytes. The particles are designed to be distinguishable by flow cytometry. Vignali, D. A. A. has described an alternative multiplex binding assay in which 64 different bead sets of microparticles are employed, each having a uniform and distinct proportion of two dyes (Vignali, D. A. A., “Multiplexed Particle-Based Flow Cytometric Assays,” J. Immunol. Meth. 243:243-255 (2000)). A similar approach involving a set of 15 different beads of differing size and fluorescence has been disclosed as useful for simultaneous typing of multiple pneumococcal serotypes (Park, M. K. et al., “A Latex Bead-Based Flow Cytometric Immunoassay Capable Of Simultaneous Typing Of Multiple Pneumococcal Serotypes (Multibead Assay),” Clin Diagn Lab Immunol. 7:486-9 (2000)). Bishop, J. E et al. have described a multiplex sandwich assay for simultaneous quantification of six human cytokines (Bishop, J. E. et al., “Simultaneous Quantification of Six Human Cytokines in a Single Sample Using Microparticle-based Flow Cytometric Technology,” Clin Chem. 45:1693-1694 (1999)).

Despite such methods for conducting the multiplexed analysis of multiple analytes (see U.S. Pat. No. 6,319,668 (Nova, et al)), a need remains for efficient methods capable of simultaneously assaying multiple different analytes. The present invention addresses this need, as well as other needs.

### Arrays

The CETs of this invention are suited to the detection of fluorophores on arrays. Often the fluorophore is conjugated to one binding pair member which is allowed to contact a second binding pair member (e.g. a complementary nucleotide) present on an addressable array. Such an interaction can be detected by contacting the array with a CET produced as described above.

Arrays of nucleic acids for use in gene expression monitoring are described in PCT WO 97/10365, the disclosure of which is incorporated herein. In one embodiment, arrays of nucleic acid probes are immobilized on a surface, wherein the array comprises more than 100 different nucleic acids and wherein each different nucleic acid is localized in a predetermined area of the surface, and the density of the different nucleic acids is greater than about 60 different nucleic acids per 1 cm².

Arrays of nucleic acids immobilized on a surface which may be used also are described in detail in U.S. Pat. No. 5,744,305, the disclosure of which is incorporated herein. As disclosed therein, on a substrate, nucleic acids with different sequences are immobilized each in a predefined area on a surface. For example, 10, 50, 60, 100, 10³, 10⁴, 10⁶, 10⁷, or 10⁸ different monomer sequences may be provided on the substrate.

The nucleic acids of a particular sequence are provided within a predefined region of a substrate, having a surface area, for example, of about 1 cm² to 10⁻¹⁰ cm². In some embodiments, the regions have areas of less than about 10⁻¹, 10⁻², 10⁻³, 10⁻⁴, 10⁻⁵, 10⁻⁶, 10⁻⁷, 10⁻⁸, 10⁻⁹, or 10⁻¹⁰ cm². For example, in one embodiment, there is provided a planar, non-porous support having at least a first surface, and a plurality of different nucleic acids attached to the first surface at a density exceeding about 400 different nucleic acids/cm², wherein each of the different nucleic acids is attached to the surface of the solid support in a different predefined region, has a different determinable sequence, and is, for example, at least 4 nucleotides in length. The nucleic acids may be, for example, about 4 to 20 nucleotides in length. The number of different nucleic acids may be, for example, 1000 or more. In the embodiment where polynucleotides of a known chemical sequence are synthesized at known locations on a substrate, and binding of a complementary nucleotide is detected, and wherein a fluorescent label is detected, detection may be implemented by directing a CET to the substrate. For example, the substrate is placed in a microscope detection apparatus for identification of locations where binding takes place. The microscope detection apparatus includes a means for detecting emitted light from the fluorophore, and means for determining a location of the fluoresced light. The means for detecting light emitted on the substrate may in some embodiments include a photon counter. The means for determining a location of the fluoresced light may include an x/y translation table for the substrate. Translation of the substrate and data collection are recorded and managed by an appropriately programmed digital computer, as described in U.S. Pat. No. 5,510,270, the disclosure of which is incorporated herein. In one embodiment, the individually isolated probes may be attached to the matrix at defined positions. These probe reagents may be attached by an automated process making use of the caged biotin methodology described in Ser. No. 07/612,671, or using photochemical reagents, see, e.g., Dattagupta et al. (1985) U.S. Pat. No. 4,542,102 and (1987) U.S. Pat. No. 4,713,326. Each individually purified reagent can be attached individually at specific locations on a substrate.

The methods and compositions described herein may be used in a range of applications including biomedical and genetic research and clinical diagnostics. Arrays of polymers such as nucleic acids may be screened for specific binding to a target, such as a complementary nucleotide, for example, in screening studies for determination of binding affinity and in diagnostic assays. In one embodiment, sequencing of polynucleotides can be conducted, as disclosed in U.S. Pat. No. 5,547,839, the disclosure of which is incorporated herein. The nucleic acid arrays may be used in many other applications including detection of genetic diseases such as cystic fibrosis, diabetes, and acquired diseases such as cancer, as disclosed in U.S. patent application Ser. No. 08/143,312, the disclosure of which is incorporated herein. Genetic mutations may be detected by sequencing by hydridization. In one embodiment, genetic markers may be sequenced and mapped using Type-IIs restriction endonucleases as disclosed in U.S. Pat. No. 5,710,000, the disclosure of which is incorporated herein.

In the present invention a microarray containing binding pair members is contacted with a CET, and the emission from fluorophore(s) on the microarray are detected and quantified. It is presumed that an automated process can contact the microarray with the CET, such that detection can be instantaneous. Additionally, an instrument containing a detection system (as described below), and an automated mechanism to deliver CET to the surface of a microarray is envisioned.

### Beads

The detection of binding pair members can also occur on beads. For the purposes of the present invention beads, microspheres, and resins are considered equivalent. Any type of bead can conceivably be used, including polystyrene, polyacrylamide, sepharose, agarose, polydivinylbenzene, silica, silica gel, amino-functionalized silica gel, alumina, or paramagnetic particles (including encapsulated paramagnetic particles). In one aspect of the invention, one binding pair member is conjugated to the bead. The second binding pair member is conjugated to a fluorophore. The conjugated said first and said second binding pair members (with their conjugated beads and fluorophore, respectively) are mixed, allowing the binding pair members to interact. The beads are then separated from the liquid phase by a method which is specific for the bead. For example, agarose beads can be separated from the surrounding liquid by centrifugation, followed by removal of the supernatant. For paramagnetic particles, application of a magnet can allow removal of the liquid phase from a vessel, while the beads remain within the vessel by virtue of their attraction to the magnetic field near the vessel. The beads can be optionally washed with a liquid, such as a buffer. The beads could also be present in a column, such that the liquid phase can be removed by gravity flow or a peristaltic pump. Detection of the interaction between the binding pair members can be accomplished by adding a CET in an appropriate solvent, followed by the detection of emission light as described below. The use of beads and the separation of beads from the liquid phase is described in Bangs, L. B., Pure & Appl. Chem., 68, 1873-1879 (1996).

### Gels

The interaction between binding pair members can also be accomplished by employing gel electrophoresis. Gel electrophoresis is common in the art, with descriptions being found in Sambrook and Russell, Molecular Cloning: A Laboratory Manual 3d ed. (2001); Kriegler, Gene Transfer and Expression: A Laboratory Manual (1990); and Ausubel et al., Current Protocols in Molecular Biology (1994).

A biomolecule can be resolved by electrophoresis on a gel. The type of gel is specific to the binding pair member resolved, and for the application of interest to the investigator. For example, polypeptides are often resolved on polyacrylamide gels containing sodium dodecyl sulfate, and polynucleotides are often resolved on agarose. However, for the purposes of the instant invention, the type of gel is not critical.

In one embodiment, one or more biomolecules are conjugated to one or more fluorophores, and said labeled molecules resolved on a gel. When different biomolecules are to be detected, preferably the fluorophores comprise different emission wavelengths. Following resolution, the gel is then exposed to a CET and detected as described below. The gel can be optionally dried prior to addition of the CET. Since different biomolecules can be labeled with different fluorophores, multiplex detection is possible (i.e. simultaneous detection of multiple different biomolecules by virtue of the different emission spectra of the fluorophores to which they are conjugated). Optionally, the biomolecules can be transferred to a second solid support, preferably a membrane (e.g. nylon or nitrocellulose, and the like). The membrane can then be contacted with a CET, such that emission of the fluorophores can be detected.

In another embodiment, a biomolecule which is not conjugated to a fluorophore is resolved on a gel, followed by contacting the gel to a second biomolecule which is conjugated to a fluorophore. The detection of the interaction between the two binding pair members is accomplished by exposing the gel to a CET, which can activate the fluorophore allowing light emission at the position of the first binding pair member, if the two binding pair members interact. Detection can be accomplished by measuring the emission light of the relative positions of the gel as described below under “Detection” (e.g. by using a CCD camera). Similarly, different biomolecules conjugated with different fluorophores (preferably containing different emission wavelengths), can be contacted with the gel, then multiple binding pair member interactions can be detected simultaneously.

In still another embodiment, one or more biomolecules (which are not conjugated to a fluorophore) can be resolved on a gel, then transferred to a second solid support which is preferably a membrane, and the membrane contacted with one or more second biomolecules which are conjugated to one or more fluorophores, respectively. The membrane can then be contacted with a CET such that multiple binding pair interactions can be detected simultaneously on the membrane.

### Detection

In a preferred embodiment, the binding pair members on a solid support are excited with a CET and the resulting fluorescence at the emission wavelength is detected.

When an array is the solid support, devices for concurrently processing multiple biological chip assays may be used as described in U.S. Pat. No. 5,545,531, the disclosure of which is incorporated herein. Methods and systems for detecting a labeled marker on a sample on a solid support, wherein the labeled material emits radiation at a wavelength that is different from the excitation wavelength, which radiation is collected by collection optics and imaged onto a detector which generates an image of the sample, are disclosed in U.S. Pat. No. 5,578,832, the disclosure of which is incorporated herein. These methods permit a highly sensitive and resolved image to be obtained at high speed. Methods and apparatus for detection of fluorescently labeled materials are further described in U.S. Pat. Nos. 5,631,734 and 5,324,633, the disclosures of which are incorporated herein. The only requirement for such detection systems with regards to the present invention are that the chemical CET be utilized to excite the fluorophore in place of the incident light source which is common in current detectors.

Detection of the fluorescence signal can utilize a confocal microscope. The microscope may be equipped with a phototransducer (e.g., a photomultiplier, a solid state array, a CCD camera, etc.) attached to an automated data acquisition system to automatically record the chemiluminescent signal produced by the fluorophore. Such automated systems are described at length in U.S. Pat. No. 5,143,854, PCT Application No. 20 92/10092, and copending U.S. Ser. No. 08/195,889 filed on Feb. 10, 1994.

### Nucleic Acid Sequencing

One widely used application for fluorescence detection is DNA sequencing. Such fluorescence can also be detected by activating the fluorophores used in sequencing with a CET. Approaches to sequencing DNA have varied widely. The Maxam-Gilbert technique for sequencing (Maxam and Gilbert, 1977, PNAS USA 74:560) involves four separate chemical cleavage reactions using the same DNA molecules. The partial or total cleavage of the DNAs, which are end-labeled, produce varying sized DNAs which are run on a gel electrophoresis apparatus. The sequence of the DNA molecule is determined from the migratory position of the bands in the gel. The dideoxy method of sequencing (Sanger et al., 1977, PNAS USA 74:5463) involves four enzymatic reactions using DNA polymerase to synthesize fragments of varying lengths due to the incorporation of a chain terminating dideoxy nucleotide into each fragment. Variations on the Sanger method comprise the use of fluorescent dye-labeled primers or nucleotide chain terminators. The reactions are then run on a gel electrophoresis apparatus. In the present invention, the sequence of the DNA molecule is determined from the migratory position of the cleaved bands in the gel, when contacted with a CET. Fluorescence emissions from the dyes are determined during exposure of the gel to a CET, allowing sequence information to be gathered based on the emission pattern.

Alternatively, sequencing methods involving the use of an exonuclease to cleave off a terminal nucleotide of a single DNA molecule have been described. Jett et al. (U.S. Pat. No. 4,962,037) describes a method wherein a complementary strand of the DNA to be sequenced is synthesized with nucleotides covalently bonded to a fluorescent dye. Then, the labeled complementary strand of the desired DNA is sequenced using exonuclease cleavage. In practice, the exonuclease cleavage is hindered by the presence of dye on each nucleotide. Ishikawa (U.S. Pat. No. 5,528,046) describes the use of monoclonal antibodies against nucleotides A, G, T or C for detecting nucleotides freed from the DNA being sequenced. The monoclonal antibody in Ishikawa may be conjugated to a light emitting reagent, particularly a luminescent enzyme, to facilitate detection of the freed nucleotide. In each of these methods, spectral properties of molecules attached to a nucleic acid are utilized to determine DNA sequence. In principle, such methods are amenable to excitation of a fluorophore with a CET, utilizing existing fluorescence detection systems.

All references and patent publications referred to herein are hereby incorporated by reference herein.

As can be appreciated from the disclosure provided above, the present invention has a wide variety of applications. Accordingly, the following examples are offered for illustration purposes and are not intended to be construed as a limitation on the invention in any way.

### EXAMPLES

### Example 1

### CETs and How to Make Them

### Bis(2,4-dinitrophenyl) Oxalate

A solution of 368.2 g. (2 moles) of 2,4 dinitrophenyl in 5 L. of benzene is dried by azeotropic distillation of 1 L. of solvent. The dried solution is cooled to 10 C under a nitrogen atmosphere and 202.4 g. (2 moles) of freshly distilled triethylamine is added. Oxalyl chloride 139.6 g. (1.1 moles) is added to this mixture during 30 minutes using a cooling bath to maintain the reaction temperature between 10-25 C. The resultant yellow slurry is stirred for three hours, then evaporated to dryness under reduced pressure, This solid after mixing with 1 L. of chloroform, is collected on a sintered glass funnel, is washed with chloroform and is dried under vacuum. Recrystallization from nitrobenzene provides 151.3 g. (35.8%) of pale yellow crystals. MP 189-192 C.

### Bis(6-carboxy-2,4,5-trichlorophenyl)oxalate

3,5,6-trichlorosalicylic acid 12.7 g. (0.05 M) and 9.3 g. (0.05 M) of dodecylamine were dissolved in 400 ml of benzene. The solution was evaporated to dryness under an argon atmosphere to obtain a white solid which was washed with 80 ml. of n-hexane to obtain 16.3 g. of a white product m.p. 118-119 C. This product was dissolved in a mixture of 160 ml of benzene and 320 ml of anhydrous ether. There solution was treated with 3.6 ml (0.0423 M) of oxalyl chloride and 10.9 ml (0.079 M) of triethylamine at 25 C. The mixture was stirred for 20 minutes and filtered. The mothor liquor was evaporated to dryness, and the residue was washed successively with 160 ml of n-hexane, and 300 ml of boiling benzene to obtain 3.0 g. (14%) of a white solid product. (m.p. 168.5-169 C)

### N-2,4,5-Trichlorophenyl)-Trifluoromethanesulfonamide

Trifluoromethanesulfonic anhydride (14.11 g. 0.05 mole) is added in portions to a stirred solution of 2,4,5-trichloroanline (9.8 g. 0.05 mole) and triethylamine (5.0 g. 0.05 mole) in methtlene chloride (50 mls) at 0 C. under a nitrogen atmosphere. The reaction mixture is stirred at 0 C for one hour upon completion of the addition, then heated to 50 C. and stirred for three hours. The white solid precipitate is separated by filtration, and the filtrate is evaporated to obtain a dark oil. Water (40 mls) is assed to the oil and the resulting residue is extracted three times with ethyl ether (50 mls). The combined ethereal extracts are then dried over anhydrous sodium sulfate. The dried extract is separated and the separated ethereal solution is evaporated to obtain 13.4 g. of crude product. Recrystallization of the crude product from methylcyclohexane gives the desired product m.p. 104-106 C.

### N,N′-Bis(2,4,5-trichlorophenyl)-N,N″-Bis(trifluoromethylsulfonyl)Oxamide

Oxalyl Chloride (1.27 g 0.01 mole) is added portionwise to a stirred solution of N-2,4,5-trichlorophenyl-trifluoromethanesulfonamide (6.2 g 0.02 mole) and triethylamine (2.0 g. 0.02 mole) in methylene chloride (50 mls) at 0 C under a nitrogen atmosphere. After the addition is completed, the reaction mixture is stirred at room temperature for four hours and then filtered. The filtrate is evaporated to obtain 2.1 g of crude product. Recrystallization of the crude product from methylcyclohexane affords the desired product m.p. 190-192 C.

### Bis-(4-chlorophenyl) Oxalyl Sulfide

Following the procedure in the above examples oxalyl chloride is added to a solution of 4-chlorothiophenol and triethylamine in methylene chloride. The mixture is stirred for a period of one hour and the mixture filtered. The filtrate is dried over anhydrous sodium sulfate, and the filtrare is evaporated to yield the product.

### Example 2

### An Assay to Determine Compatibility of a CET and Fluorophore

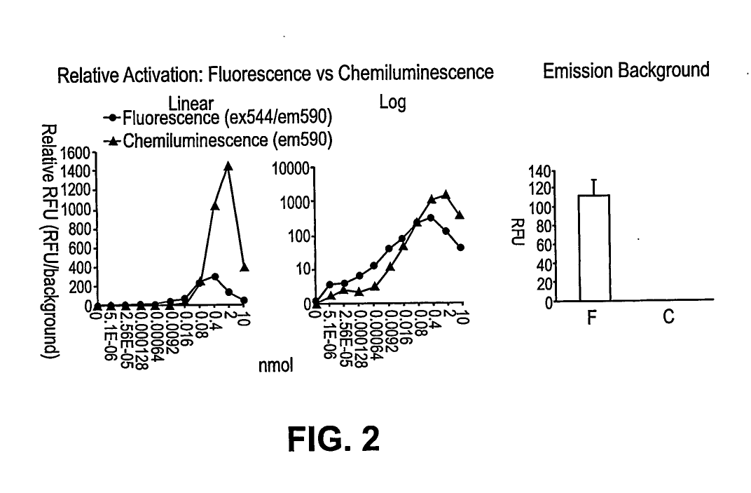

A CET (2,4,5-trichlorocarboxyphenyl oxalate) at 100 mM in a solution of 1 mM sodium salicylate and 100 mM H₂O₂ (hydrogen peroxide) in dibutyl phthalate was incubated with 10 mM of rhodamine B dissolved in H₂O in a final volume of 1001 in a microtiter plate. A red glow was seen to emit from the well indicating compatibility of the dye and CET. For further quantification, the CET was incubated with increasing concentrations of rhodamine B, and emission was measured at 590 mm on a microplate reader (f-max, Molecular Devices, Sunnyvale, Calif.). The fluorescence intensity was compared to an equal concentration of rhodamine B activated by incident light at 544 nm on the plate reader. Results of the experiment are shown in FIG. 2, left and middle panels. The background signal is illustrated in the right panel for the CET and fluorescence activation.

### Example 3

Protein based assay. A nuclear extract from HeLa cells is obtained according to published methods (Rathmell and Chu Mol. Cell. Biol. 14: 4741-4748(1994a)). The extract is resolved by SDS-PAGE and transferred to nitrocellulose membrane as described (Smider, et al. Science 266: 288-291 (1994)). Antibodies to Ku70 and Ku86 are obtained from Santa Cruz Antibody, and are conjugated to TAMRA and ROX, respectively, according to the manufacturers instructions (Molecular Probes, Eugene, Oreg.). Following blocking with 2% dried milk in phosphate buffered saline, the antibodies are added to the membrane at a 1:500 dilution and incubated for 1 hour at 37° C. The membrane is washed in wash buffer (0.05% tween-20 in 10 mM Tris pH 7.4, 1 mM EDTA). The membrane is then contacted with 10 ml of a CET (Bis(2,4-dinitrophenyl) oxalate) at 100 mM in a solution of 1 mM sodium salicylate and 100 mM H₂O₂ in dibutyl phthalate.

Nucleic acid based assay. The primer pairs (ROX)-TACAGGGTGGGTTTACC (IgM secretory region), GTTTGCAAG TGTCCAGTGT (human VH3), and GTTTGCAAGTGTCCAGTGT, (R6G)-TGAGGAGACGGTGACCAGGGT (human JH) are used to amplify human spleen cDNA (Stratagene, LaJolla, Calif.) to obtain full length IgM (first primer pair) and V-region (second primer pair) antibody gene PCR products (McCafferty, et al. IRL Press Oxford, UK (1996)). ROX and R6G rhodamine based fluorophores are attached at the 5′ end of the respective primers. Amplification is carried out using standard PCR conditions, 100 ng of cDNA as template, and pfu polymerase as described (Griffin and Griffith CRC Press Boca Raton (1994)). The amplified products are resolved on a 1% agarose gel without staining. The gel is then rinsed in TAE buffer, and submerged in a solution containing CET reagent (Bis(6-carboxy-2,4,5-trichlorophenyl)oxalate and 100 mM H₂O₂) and imaged with a CCD camera.

1. A non-enzymatic, non-fluorescent, chemiluminescent method of detecting the interaction between two binding pair members by:
attaching a fluorophore to one of the two binding partner members;
immobilizing the unlabeled binding pair member to a solid support;
allowing the two binding pair members to bind to each other; and
contacting the binding pair members with a solution comprising a chemical-energy transferring composition under conditions that stimulate the release of light energy from the fluorophore to allow detection of the interaction between the two binding pair members.

2. A method of claim 1 wherein the chemical-energy transferring composition comprises an oxalic type compound.

3. A method of claim 2 wherein the oxalic type compound is selected from: an oxalate ester, an oxalic thioester, an oxalate amide, a phosphate containing oxalic type compound.

4. A method of claim 3, wherein the oxalic type compound contains electronegative substituents.

5. A method of claim 4, wherein the electronegative substituents are halogen atoms.

6. A method of claim 5, wherein the halogen atom is chlorine.

7. A method of claim 3 wherein the fluorophores are selected from one of the following groups: xanthenes, coumarins, benzimides, phenanthridines, acridines, cyanines, bodipy dyes, carbazole dyes, phenoxazine dyes, porphyrins, quinolines, polycyclic aromatic hydrocarbons containing at least three fused rings, and quantum dots.

8. A method of claim 7, wherein the fluorophore comprises a parent heteroaromatic ring system.

9. A method of claim 7, wherein the fluorophore comprises a parent xanthene ring.

10. A method of claim 9, wherein the fluorophore comprises a rhodamine-type parent xanthene ring or a fluorescein-type xanthene ring.

11. A method of claim 7, wherein the fluorophore comprises a cyanine dye.

12. A method of claim 10, wherein the fluorophore comprises a rhodamine dye or a fluorescein dye.

13. A method of claim 1 where the binding pair members are selected from the group consisting of: an antibody and antigen, two complementary nucleic acids, a protein and nucleic acid, a virus and host receptor, and a hormone and its cognate receptor.

14. A method of claim 1 where the solid support is selected from the group consisting of an addressed microarray, a bead, a gel and a transparent surface.

15. A method of sequencing a target nucleic acid by:
i. copying the target nucleic acid using a polymerase and nucleotide triphosphates;
ii. randomly terminating polymerase activity using four polymerase blocking nucleotide inhibitors bearing a fluorophore specific for that inhibitor where the inhibitors are present in concentrations able to yield polymerase products terminated at different lengths;
iii. size fractionating the polymerase products in a gel; and,
iv. contacting the products with a solution comprising an oxalic type compound under conditions that stimulate the release of light energy from the fluorophore to allow detection of the product within the gel to sequence the nucleic acid.

16. A non-enzymatic non-fluorescent chemiluminescent system for detecting the interaction between two binding pair members said system comprising an immobilized binding pair member ad a non-immobilized binding pair member labeled with a fluorophore and a solution comprising a chemical energy transferring composition that is photo-reactive with the fluorophore.

17. A system of claim 16 wherein the chemical-energy transferring composition comprises an oxalic type compound.

18. A system of claim 17 wherein the oxalic type compound is selected from: an oxalate ester, an oxalic thioester, an oxalate amide, a phosphate containing oxalic type compound.

19. A system of claim 18, wherein the oxalic type compound contains electronegative substituents.

20. A system of claim 19, wherein the electronegative substituents are halogen atoms.

21. A system of claim 20, wherein the halogen atom is chlorine.

22. A system of claim 18 wherein the fluorophores are selected from one of the following groups: xanthenes, coumarins, benzimides, phenanthridines, acridines, cyanines, bodipy dyes, carbazole dyes, phenoxazine dyes, porphyrins, quinolines, polycyclic aromatic hydrocarbons containing at least three fused rings, and quantum dots.

23. A system of claim 22, wherein the fluorophore comprises a parent heteroaromatic ring system.

24. A system of claim 22, wherein the fluorophore comprises a parent xanthene ring.

25. A system of claim 24, wherein the fluorophore comprises a rhodamine-type parent xanthene ring or a fluorescein-type xanthene ring.

26. A system of claim 22, wherein the fluorophore comprises a cyanine dye.

27. A system of claim 25, wherein the fluorophore comprises a rhodamine dye or a fluorescein dye.

28. A system of claim 16 where the binding pair members are selected from the group consisting of: an antibody and antigen, two complementary nucleic acids, a protein and nucleic acid, a virus and host receptor, and a hormone and its cognate receptor.

29. A system of claim 16 where the solid support is selected from the group consisting of an addressed microarray, a bead, a gel and a transparent surface.

30. A system comprising a nucleic acid labeled with a fluorophore where the nucleic acid is in a gel and where the gel is infused with solution comprising an a chemical energy transferring composition that is photo-reactive with the fluorophore.

31. A system of claim 30 wherein the chemical-energy transferring composition comprises an oxalic type compound.

32. A system of claim 31 wherein the oxalic type compound is selected from: an oxalate ester, an oxalic thioester, an oxalate amide, a phosphate containing oxalic type compound.

33. A system of claim 32, wherein the oxalic type compound contains electronegative substituents.

34. A system of claim 33, wherein the electronegative substituents are halogen atoms.

35. A system of claim 34, wherein the halogen atom is chlorine.

36. A system of claim 32 wherein the fluorophores are selected from one of the following groups: xanthenes, coumarins, benzimides, phenanthridines, acridines, cyanines, bodipy dyes, carbazole dyes, phenoxazine dyes, porphyrins, quinolines, polycyclic aromatic hydrocarbons containing at least three fused rings, and quantum dots.

37. A system of claim 36, wherein the fluorophore comprises a parent heteroaromatic ring system.

38. A system of claim 36, wherein the fluorophore comprises a parent xanthene ring.

39. A system of claim 38, wherein the fluorophore comprises a rhodamine-type parent xanthene ring or a fluorescein-type xanthene ring.

40. A system of claim 36, wherein the fluorophore comprises a cyanine dye.

41. A system of claim 39, wherein the fluorophore comprises a rhodamine dye or a fluorescein dye.

42. A system of claim 30 where the binding pair members are selected from the group consisting of: an antibody and antigen, two complementary nucleic acids, a protein and nucleic acid, a virus and host receptor, and a hormone and its cognate receptor.

43. A system of claim 30 where the solid support is selected from the group consisting of an addressed microarray, a bead, a gel and a transparent surface.

44. A system for detecting a biological composition comprising a biological composition labeled with a fluorophore where the composition is bound to a solid support and where the solid support is contacted with a solution comprising an oxalic type compound and a hydroperoxide.

45. A system of claim 44 wherein the chemical-energy transferring composition comprises an oxalic type compound.

46. A system of claim 45 wherein the oxalic type compound is selected from: an oxalate ester, an oxalic thioester, an oxalate amide, a phosphate containing oxalic type compound.

47. A system of claim 46, wherein the oxalic type compound contains electronegative substituents.

48. A system of claim 47, wherein the electronegative substituents are halogen atoms.

49. A system of claim 48, wherein the halogen atom is chlorine.

50. A system of claim 46 wherein the fluorophores are selected from one of the following groups: xanthenes, coumarins, benzimides, phenanthridines, acridines, cyanines, bodipy dyes, carbazole dyes, phenoxazine dyes, porphyrins, quinolines, polycyclic aromatic hydrocarbons containing at least three fused rings, and quantum dots.

51. A system of claim 50, wherein the fluorophore comprises a parent heteroaromatic ring system.

52. A system of claim 50, wherein the fluorophore comprises a parent xanthene ring.

53. A system of claim 52, wherein the fluorophore comprises a rhodamine-type parent xanthene ring or a fluorescein-type xanthene ring.

54. A system of claim 50, wherein the fluorophore comprises a cyanine dye.

55. A system of claim 53, wherein the fluorophore comprises a rhodamine dye or a fluorescein dye.

56. A system of claim 44 where the binding pair members are selected from the group consisting of: an antibody and antigen, two complementary nucleic acids, a protein and nucleic acid, a virus and host receptor, and a hormone and its cognate receptor.

57. A system of claim 44 where the solid support is selected from the group consisting of an addressed microarray, a bead, a gel and a transparent surface.

58. A chemical energy transferring mixture comprising:
a) an oxalic type compound of the formula Z(CO)₂Z
b) a peroxide component
c) a biomolecule

59. The mixture of claim 58, wherein Z contains one of the following atoms: an oxygen, a sulfur, a nitrogen, a phosphorus.

60. The mixture of claim 59, wherein the oxalic type compound contains electronegative substituents.

61. The mixture of claim 60, wherein the electronegative substituents are halogens.

62. The mixture of claim 61, wherein the electronegative substituents are chlorine.

63. The mixture of claim 62, wherein the oxalic type compound is an oxalate ester.

64. The mixture of claim 58, wherein the biomolecule is selected from: a polynucleotide, an oligonucleotide, a peptide, a polypeptide, a polysaccharide.

65. The mixture of claim 58, wherein the peroxide component is hydrogen peroxide.
